(19) United States
(12) Patent Application Publication
(10) Pub. No.: US 2022/0249096 A1
(43) Pub. Date: Aug. 11, 2022

(54) AUTOMATIC COMPRESSION CIRCULAR ANASTOMOSIS DEVICE AND CONTROL METHOD THEREOF

(71) Applicant: PUSAN NATIONAL UNIVERSITY INDUSTRY-UNIVERSITY COOPERATION FOUNDATION, Busan (KR)

(72) Inventors: Gyung Mo SON, Yangsan-si (KR); Kwang Ryul BAEK, Busan (KR); Sang Ho PARK, Busan (KR); Jin Seok KIM, Busan (KR)

(73) Assignee: PUSAN NATIONAL UNIVERSITY INDUSTRY-UNIVERSITY COOPERATION FOUNDATION, Busan (KR)

(21) Appl. No.: 17/589,069

(22) Filed: Jan. 31, 2022

(30) Foreign Application Priority Data
Feb. 8, 2021 (KR) 10-2021-0017499

(51) Int. Cl. A61B 17/115

(52) U.S. Cl. CPC A61B 17/1155; A61B 2017/0046; A61B 17/1152

(57) ABSTRACT

A control method of an automatic compression circular anastomosis device, includes: correcting a zero point when an operation switch provided in a handle unit is turned on, by a control unit; controlling a motor to compress a tissue in a direction reaching a minimum distance at which a distance between a top surface of an anastomosis unit and a bottom surface of an anvil is minimized, by the control unit; measuring a real-time pressure applied to the tissue and a real-time distance between the top surface of the anastomosis unit and the bottom surface of the anvil in real-time, by the control unit; and controlling, when the real-time pressure and the real-time distance reach a set pressure and a set distance Dᵣ stored in advance, the anastomosis unit to cut unnecessary parts of the tissue and anastomose the tissue with a plurality of staples, by the control unit.

### CROSS-REFERENCE TO RELATED APPLICATION

This application claims priority to Korean Patent Application No. 10-2021-0017499 filed on Feb. 8, 2021, which is hereby incorporated by reference in its entirety.

### BACKGROUND

The present invention relates to an automatic compression circular anastomosis device and a control method thereof, and more particularly, to an automatic compression circular anastomosis device that implements automatic anastomosis compression by limiting compression pressure and determining a compression stop time during anastomosis surgery, and a control method thereof.

Laparoscopic surgery is a surgical method of making several small incisions in the abdomen and inserting various surgical instruments therein. From the viewpoint of a patient, since the size of incision is small, surgical scars remain small and look aesthetically good, and pains caused by the incision are much less severe. In addition, since it shows a quick recovery rate, the period of staying in the hospital is shorter than the case of making open surgery, and has an advantage of returning to daily life quickly.

Although the success rate of preserving the anal of a rectal cancer patient has been improved remarkably with development of double staple anastomosis techniques using a circular anastomosis device in laparoscopic surgery, about 10 to 20% of surgical patients suffer from anastomotic complications.

Inadequate tissue compression is one of major causes of the anastomotic complications. Since conventional manual circular anastomosis devices operate according to the operator's clinical experiences and intuitive feelings, each operator performs tissue compression with different pressures, and therefore, in this case, complications such as anastomotic necrosis, ischemia and the like due to over-compression and complications such as anastomotic leakage and the like due to under-compression may occur.

Patent document 1 relates to an automatic purse-string suture device for automatically performing purse-string suture before using a circular anastomosis device, and although a plurality of stapler iron cores can be easily put into the surface to fasten a target area with a string, there is a disadvantage in that occurrence of anastomotic complications due to over-compression or under-compression cannot be significantly reduced since the pressure applied to the target area cannot be controlled.

Patent document 2 relates to a circular anastomosis device, and although a body unit including a housing having an integrally formed handle and a rotatable trigger formed in the central lower portion of the body unit are provided so that a user may hold and pull the trigger in the direction of the handle while supporting the handle with one hand, this also has a disadvantage in that occurrence of anastomotic complications due to over-compression or under-compression cannot be significantly reduced since the pressure applied to the target area cannot be controlled.

Accordingly, it needs to establish a quantified and digitized tissue anastomosis device and method that can improve safety and significantly reduce occurrence of anastomotic complications during laparoscopic surgery.
(Patent Document 1) Korea patent registration No. 10-2020-0088632
(Patent Document 2) Korea patent registration No. 10-2139495

### SUMMARY

Therefore, the present invention has been made in view of the above problems, and it is an object of the present invention to provide an automatic compression circular anastomosis device comprising an anvil, a separation coupling unit, an anastomosis unit, a shaft, a handle unit, a body, and a control unit, which can reduce damage to a compression surface between tissues that a surgeon does not recognize with naked eyes and reduce the prevalence rate of complications by preventing over-compression or under-compression of the tissues when a perforated tissue is anastomosed during laparoscopic surgery, and to obtain a control method of the automatic compression circular anastomosis device, which can grasp a real-time pressure Pₙ applied to the tissue and a real-time distance Dₙ between the top surface of the anastomosis unit and the bottom surface of the anvil.

To accomplish the above object, according to one aspect of the present invention, there is provided an automatic compression circular anastomosis device comprising: an anvil formed in a conical shape to be easily inserted into a perforated tissue; a separation coupling unit having a central axis formed in a direction from the bottom surface of the anvil toward the outside; an anastomosis unit for pressing, when the tissue is positioned around the separation coupling unit, both sides of the tissue together with the bottom surface of the anvil, cutting unnecessary parts of the tissue, and anastomosing the tissue with a plurality of staples; a shaft bent to be easily inserted into the perforated tissue and provided with a screw gear for converting the rotational force of the motor into an axial linear movement to allow the separation coupling unit coupled to the anastomosis unit to move linearly to transfer power; a handle unit formed to accommodate one side of the shaft and gripped by a user to generate a pressing force by an external force of the user; a body including a bearing having a shaft hole, the motor having a power transmission shaft passing through the shaft hole on one side and generating a rotational force, a couple ring locked and fixed to the power transmission shaft between one side of the bearing and the motor not to generate eccentricity, a power transmission adapter located on the other side of the bearing to transmit the rotational force of the motor to the screw gear, an encoder having a rotating shaft coupled on the other side of the motor and measuring an amount of rotation of the rotating shaft, and an emergency switch for immediately cutting off the power applied to a control unit; and the control unit including a current sensor for measuring a current value according to the rotational force of the motor, a pressure calculation unit for calculating a real-time pressure Pₙ applied to the tissue using the current value, and a distance calculation unit for calculating a distance between the top surface of the anastomosis unit and the bottom surface of the anvil using the rotation amount measured by the encoder, and controlling the motor according to a set pressure Pᵣ and a set distance Dᵣ stored in advance.

To accomplish the above object, according to another aspect of the present invention, there is provided a control method of an automatic compression circular anastomosis device, the method comprising: a correction step of correcting a zero point when an operation switch provided in a handle unit is turned on, by a control unit; a motor control step of controlling a motor to compress a tissue in a direction reaching a minimum distance D₀ at which the distance between the top surface of an anastomosis unit and the bottom surface of an anvil is minimized, by the control unit; a real-time pressure Pₙ and distance Dₙ measurement step of measuring a real-time pressure Pₙ applied to the tissue and a real-time distance Dₙ between the top surface of the anastomosis unit and the bottom surface of the anvil in real-time, by the control unit; and an anastomosis unit control step of controlling, when the real-time pressure Pₙ and the real-time distance Dₙ reach a set pressure Pᵣ and a set distance Dᵣ stored in advance, the anastomosis unit to cut unnecessary parts of the tissue and anastomose the tissue with a plurality of staples, by the control unit.

### BRIEF DESCRIPTION OF THE DRAWINGS

FIG. 1 is a view showing pressure deviation among medical staff members when tissue compression is performed using a conventional manual compression circular anastomosis device.

FIG. 2 is a view showing that real-time pressure Pₙ applied to a tissue over time is limited by a control unit having a set pressure Pᵣ stored in advance according to an embodiment of the present invention.

FIG. 3 is a view showing the configuration of an automatic compression circular anastomosis device of the present invention.

FIG. 4 is a side view showing an automatic compression circular anastomosis device according to an embodiment of the present invention.

FIG. 5 is a detailed side view showing the body according to an embodiment of the present invention.

FIG. 6 is a flowchart illustrating a control method of an automatic compression circular anastomosis device of the present invention.

FIG. 7 is a detailed flowchart illustrating a correction step according to an embodiment of the present invention.

### DETAILED DESCRIPTION

Although general terms widely used currently are selected as the terms used in this specification as much as possible while considering the functions in the present invention, the terms may vary according to the intention of those skilled in the art, judicial precedents, advent of new technologies, or the like. In addition, in a specific case, there may be terms arbitrarily selected by the applicant, and in this case, the meaning will be described in detail in the description of the corresponding invention. Therefore, the terms used in the present invention should be defined based on the meaning of the terms and the overall content of the present invention, rather than simply based on the names of terms.

Unless otherwise defined, all terms used herein, including technical or scientific terms, have the same meaning as commonly understood by those skilled in the art. Terms such as those defined in a commonly used dictionary should be interpreted as having a meaning consistent with the meaning in the context of related arts, and should not be interpreted as an ideal or excessively formal meaning unless explicitly defined in this application.

Automatic Compression Circular Anastomosis Device

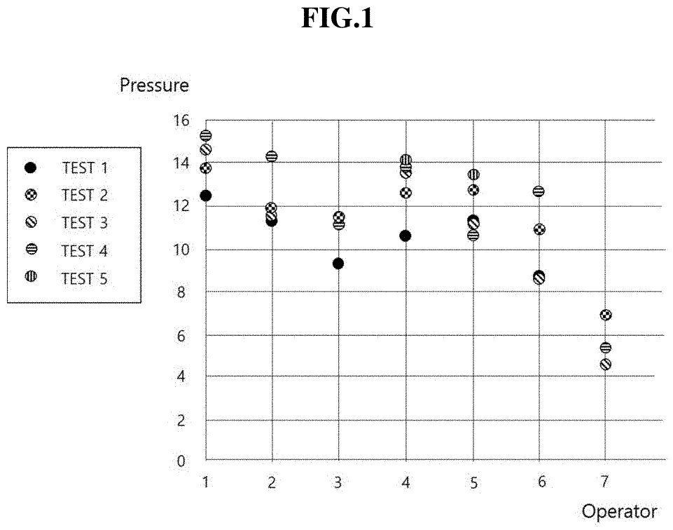

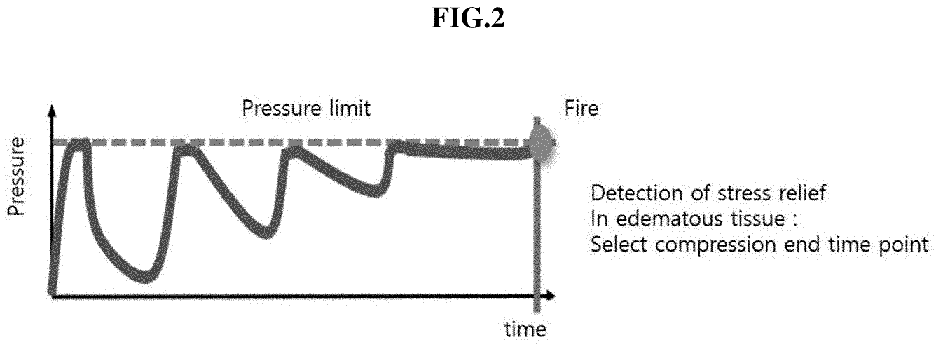

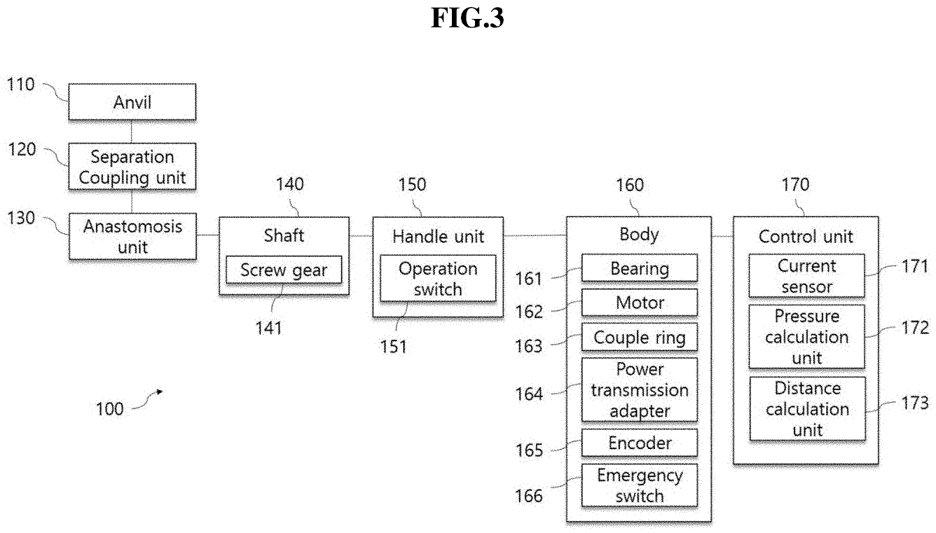

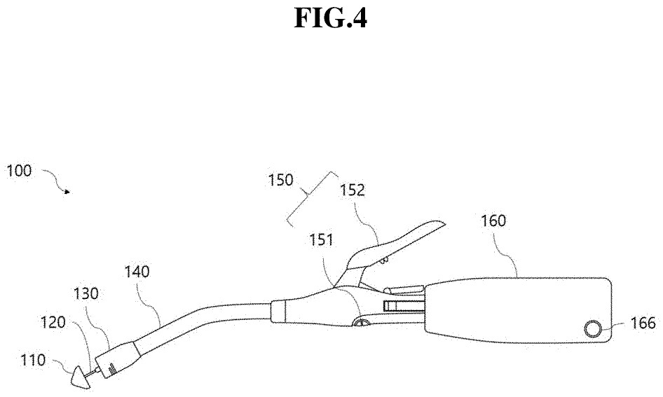

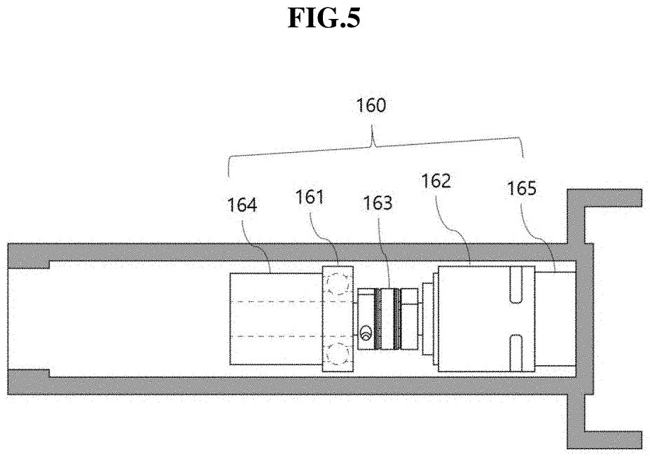

Hereinafter, an embodiment according to the present invention will be described in detail with reference to the accompanying drawings. FIG. 1 is a view showing pressure deviation among medical staff members when tissue compression is performed using a conventional manual compression circular anastomosis device. FIG. 2 is a view showing that real-time pressure Pₙ applied to a tissue over time is limited by a control unit 170 having a set pressure Pᵣ stored in advance according to an embodiment of the present invention. FIG. 3 is a view showing the configuration of an automatic compression circular anastomosis device 100 of the present invention. FIG. 4 is a side view showing an automatic compression circular anastomosis device 100 according to an embodiment of the present invention. FIG. 5 is a detailed side view showing the body 160 according to an embodiment of the present invention.

First, referring to FIG. 1, it can be confirmed that when pressure is applied to a tissue using an anastomosis device manually operated in the prior art, the pressure is different from user to user, and the pressure is different from trial to trial although the user is the same. The present invention has been devised based on this concept and provides an automatic compression circular anastomosis device and a control method thereof to apply a constant pressure to a perforated tissue regardless of a user or a trial. In addition, the present invention may anastomose a perforated tissue after previously setting an upper limit of the applied pressure and automatically determining a compression end time point so that the pressure applied to the tissue is not over-compressed over time as shown in FIG. 2.

Referring to FIGS. 3 to 4, the automatic compression circular anastomosis device 100 of the present invention includes an anvil 110, a separation coupling unit 120, an anastomosis unit 130, a shaft 140, a handle unit 150, a body 160, and a control unit 170.

More specifically, the anvil 110 is formed in a conical shape to be easily inserted into a perforated tissue. The separation coupling unit 120 has a central axis formed in a direction from the bottom surface of the anvil 110 toward the outside. When the tissue is positioned around the separation coupling unit 120, the anastomosis unit 130 presses both sides of the tissue together with the bottom surface of the anvil 110, cuts unnecessary parts of the tissue, and anastomoses the tissue with a plurality of staples.

Here, the anastomosis unit 130 may have a hollow cylinder formed on the central axis to accommodate one side of the separation coupling unit 120. Accordingly, the anastomosis unit 130 and the separation coupling unit 120 may be easily coupled and separated to have an effect of cleaning and sterilizing the parts directly contacting the tissue.

On the other hand, the automatic compression circular anastomosis device 100 of the present invention may perform vertical or linear movement to adjust the distance between the top surface of the anastomosis unit 130 and the bottom surface of the anvil 110. At this point, the automatic compression circular anastomosis device 100 may further include a protruding stopper 121 convexly formed to protrude on the outer surface of the separation coupling unit 120 to limit further linear movement in the direction of separating the separation coupling unit 120 after the separation coupling unit 120 is accommodated inside the hollow cylinder of the anastomosis unit 130, and a receiving groove stopper 131 concavely formed on the inner surface of the hollow cylinder of the anastomosis unit 130.

In addition, in the vertical movement or linear movement of adjusting the distance between the top surface of the anastomosis unit 130 and the bottom surface of the anvil 110, the distance that makes the distance between the top surface of the anastomosis unit 130 and the bottom surface of the anvil 110 minimum is the minimum distance D₀ and the distance that makes the distance maximum is the maximum distance D₁. The initial state of accommodating the separation coupling unit 120 inside the hollow cylinder of the anastomosis unit 130 is a state that maintains the maximum distance D₁.

Next, the shaft 140 is bent to be easily inserted into the perforated tissue, is provided with a screw gear 141 for converting the rotational force of the motor 162 into an axial linear movement to allow the separation coupling unit 120 coupled to the anastomosis unit 130 to move linearly, and transfers power to the anvil 110 and the separation coupling unit 120.

The automatic compression circular anastomosis device 100 is a device that anastomoses a perforated tissue most desirably during laparoscopic surgery, and the shaft 140 may be provided to have a predetermined length and to be bent at a predetermined angle at an arbitrary point.

Next, the handle unit 150 is formed to accommodate one side of the shaft 140 and gripped by a user, and a pressing force is generated by the external force of the user.

Meanwhile, the handle unit 150 may further include an operation switch 151, and when the operation switch 151 is turned on, power may be applied to the control unit 170. In addition, the control unit 170 applied with power may correct the zero point to measure real-time pressure and distance more accurately. In addition, the control unit 170 may correct the zero point at the first operation after the automatic compression circular anastomosis unit 100 is manufactured, or correct the zero point at the first operation after the operation switch 151 of the handle unit is turned on.

In addition, the motor 162 may operate when sufficient pressure is applied to an upper handle 152 by the user after the zero point is corrected. Most preferably, the handle unit 150 may be provided with the upper handle 152 connected to the upper portion of the handle unit 150 by a hinge, and pressure may be applied to the upper handle 152 after the entire handle unit 150 is gripped by the user.

This is to compensate for decrease in the pressure applied to the handle when the user's gripping strength decreases in the case of a conventional device having a handle connected to the lower portion by a hinge or a trigger-shaped handle, and as the handle unit 150 of the present invention is provided with the upper handle 152, user's weight may be transferred, and there is an effect of more constantly maintaining the pressure applied to the upper handle 152 with ease.

Next, the body 160 includes, in a housing, a bearing 161, a motor 162, a couple ring 163, a power transmission adapter 164, an encoder 165, and an emergency switch. Describing more specifically with reference to FIG. 5, the bearing 161 has a shaft hole, and this is not to move the housing and the components in the housing and not to generate friction.

Next, the motor 162 has a power transmission shaft passing through the shaft hole on one side and generates rotational force. That is, the motor 162 may generate the rotational force by rotating the power transmission shaft when power is applied or when a control signal directing to operate is received from the control unit 170.

Next, the couple ring 163 is locked and fixed to the power transmission shaft between one side of the bearing 161 and the motor 162 not to generate eccentricity. That is, the couple ring 163 capable of fixing the power transmission shaft of the motor 162 is provided to solve the problem that a constant rotational force may not be transmitted to the central axis of the screw gear 141.

Next, the power transmission adapter 164 is located on the other side of the bearing 161 to transmit the rotational force of the motor 162 to the screw gear 141. In other words, the couple ring 163, the bearing 161, and the power transmission adapter 164 may be sequentially located on the power transmission shaft as shown in FIG. 5, and the power transmission adapter 164 may be provided to meet the specification of connecting the power transmission shaft to the screw gear 141 in the shaft 140. That is, a groove for accommodating the power transmission shaft may be provided on one side of the power transmission adapter 164, and a groove for accommodating the screw gear 141 may be provided on the other side.

Next, the encoder 165 has a rotating shaft coupled on the other side of the motor 162 and measures the amount of rotation of the rotating shaft. Generally, an encoder is a device used to measure the rotation speed of a rotating object, and it may precisely measure the direction and the number of rotations of a rotating object and may also measure the rotated position of the rotating object. At this point, resolution is the most important factor among the performance factors of the encoder. The higher the resolution, the finer the rotation speed and position of the shaft, and a signal that is generated once per rotation of the encoder may be output as many as the number of resolutions.

That is, the encoder 165 is most preferably an incremental encoder, and the rotating shaft is coupled on the other side of the motor 162 and may output pulses in proportion to the rotation amount of the rotating shaft.

Next, the emergency switch 166 may be provided in a shape protruding from the surface on one side of the body 160 and connected to the control unit 170. This immediately cuts off the power applied to the control unit 170 when a failure occurs in the motor 162. Accordingly, secondary damage that may occur due to the failure of the motor 162 can be prevented.

Next, the control unit 170 includes a current sensor 171, a pressure calculation unit 172, and a distance calculation unit 173 and controls the motor 162 according to a set pressure Pᵣ and a set distance Dᵣ stored in advance.

More specifically, the current sensor 171 measures a current value according to the rotational force of the motor 162. Generally, when power is applied, current flows in the device, and when the motor 162 is driven, the current value changes. That is, the current sensor 171 is to check the change in the current value.

Next, the pressure calculation unit 172 calculates a real-time pressure Pₙ applied to the tissue using the current value. That is, the pressure calculation unit 172 may calculate a difference value between the real-time current Iₙ measured by the current sensor 171 and the driving current Iₘ and then calculate the real-time pressure Pₙ based on a previously stored current-rotation force-pressure proportion relation equation. The current-rotation force-pressure proportion relation equation is as shown in [Equation 1] and [Equation 2], and will be described below in more detail.

Next, the distance calculation unit 173 calculates the distance between the top surface of the anastomosis unit 130 and the bottom surface of the anvil 110 using the rotation amount measured by the encoder 165.

Meanwhile, the distance calculation unit 173 may obtain the number of pulses generated in proportion to the rotation amount. In addition, the distance calculation unit 173 may determine the real-time distance Dₙ using the number of pulses and the gear ratio of the screw gear 141. The real-time distance Dₙ may be calculated using [Equation 3] shown below, and will be described below in more detail.

Control Method of Automatic Compression Circular Anastomosis Device

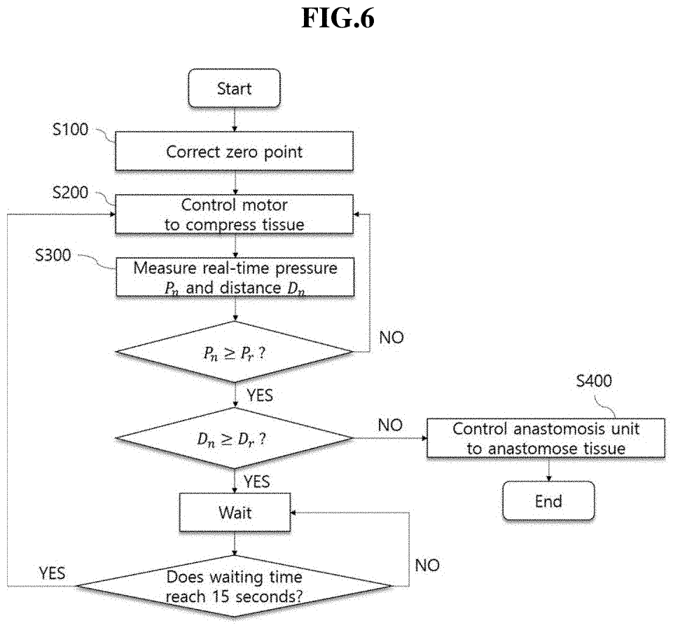

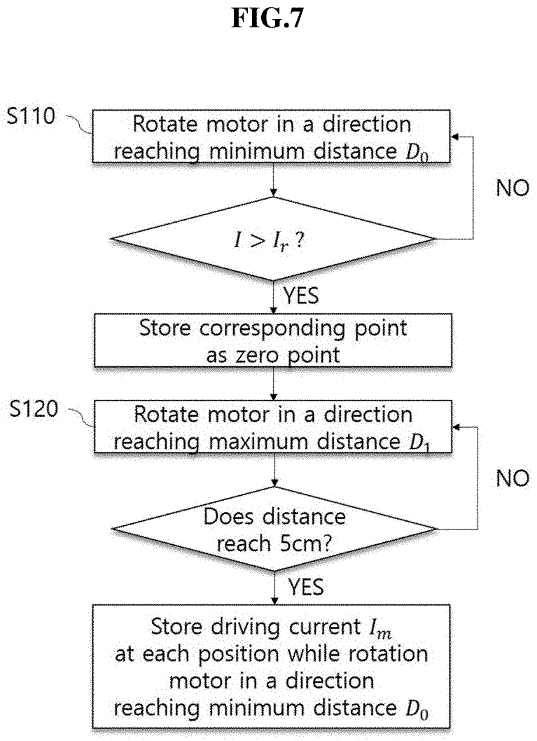

Hereinafter, an embodiment according to the present invention will be described in detail with reference to the accompanying drawings. FIG. 6 is a flowchart illustrating a control method of an automatic compression circular anastomosis device 100 of the present invention. FIG. 7 is a detailed flowchart illustrating a correction step S100 according to an embodiment of the present invention.

First, referring to FIG. 6, the control method of the automatic compression circular anastomosis device 100 of the present invention includes a correction step S100, a motor control step S200, a real-time pressure Pₙ and distance Dₙ measurement step S300, and an anastomosis unit control step S400.

More specifically, at the correction step S100, when the operation switch 151 provided in the handle unit 150 is turned on, the zero point is corrected by the control unit 170.

Describing in more detail with reference to FIG. 7, the correction step S100 may include a zero-point storage step S110, and a driving current Iₘ measurement step S120

At the zero-point storage step S110, when the motor 162 rotates in a direction reaching the minimum distance D₀, and a current value I measured by the current sensor 171 exceeds a preset current value Iᵣ, a corresponding current value I and a corresponding point may be stored as a zero-point current I₀ and a zero point by the control unit 170.

Most preferably, at the zero-point storage step S110, a position where the current value I measured by the current sensor 171 rapidly changes may be stored as the zero point. That is, the zero-point current I₀ is a current value existing as a base. At this point, the zero-point storage step S110 may be repeated until the position where the current value I measured by the current sensor 171 rapidly changes is found. That is, the zero point and the zero-point current I₀ of the automatic compression circular anastomosis device 100 may be determined at the zero-point storage step S110.

Next, at the driving current Iₘ measurement step S120, a driving current Iₘ is measured by the control unit 170 at each point while the motor 162 rotates in a direction reaching the minimum distance D₀ again after the motor 162 rotates as much as a predetermined distance in a direction reaching the maximum distance D₁ at which the distance between the top surface of the anastomosis unit 130 and the bottom surface of the anvil 110 is maximized at the zero point.

Meanwhile, the distance between the top surface of the anastomosis unit 130 and the bottom surface of the anvil 110 at the zero point may be within a range of 3 to 6 Cm, and most preferably 5 Cm. In other words, the minimum distance D₀ is the distance at which the top surface of the anastomosis unit 130 and the bottom surface of the anvil 110 reach the zero point, and the maximum distance D₁ between the top surface of the anastomosis unit 130 and the bottom surface of the anvil 110 may be 5 Cm. In addition, at the driving current Iₘ measurement step S120, the driving current Iₘ may be measured at each point within a range of 5 Cm from the zero point.

Next, at the motor control step S200, the motor 162 is controlled by the control unit 170 to compress a tissue in a direction reaching the minimum distance D₀ at which the distance between the top surface of the anastomosis unit 130 and the bottom surface of the anvil 110 is minimized.

That is, when the motor 162 is controlled at the motor control step S200, other components connected or coupled to the motor 162 may move together. The power transmission shaft of the motor 162 is accommodated on one side of the power transmission adapter 164, and the screw gear 141 is accommodated on the other side of the power transmission adapter 164. In addition, since the screw gear 141 converts the rotational motion of the motor 162 into an axial linear movement, the shaft 140 including the screw gear 141 may allow the anvil 110 and the separation coupling unit 120 to linearly move in the axis direction. Accordingly, the anvil 110 and the separation coupling unit 120 may move linearly in a direction reaching the minimum distance D₀ at which the distance between the top surface of the anastomosis unit 130 and the bottom surface of the anvil 110 is minimized, and thus the tissue may be compressed.

Next, at the real-time pressure Pₙ and distance Dₙ measurement step S300, a real-time pressure Pₙ applied to the tissue and a real-time distance Dₙ between the top surface of the anastomosis unit 130 and the bottom surface of the anvil 110 are measured in real-time by the control unit 170.

Referring to FIG. 6 again, at the real-time pressure Pₙ and distance Dₙ measurement step S300, whether the real-time pressure Pₙ reaches a set pressure Pᵣ stored in advance in the control unit 170 may be confirmed. When the real-time pressure Pₙ reaches the set pressure Pᵣ, whether the real-time distance Dₙ is greater than or equal to the set distance Dᵣ may be confirmed. At this point, when the real-time pressure Pₙ does not reach the set pressure Pᵣ, the process may return to the motor control step S200, and the motor 162 is driven to compress the tissue until the real-time pressure Pₙ reaches the set pressure Pᵣ.

In addition, at the real-time pressure Pₙ and distance Dₙ measurement step S300, when the real-time distance Dₙ is equal to or greater than the set distance Dᵣ, the motor 162 may be stopped and wait. This is a process of waiting for sufficient drainage of the intercellular fluid in the tissue in the process of repeating compression and waiting. That is, the motor 162 waits until the waiting time T reaches a preset time, and when the waiting time T reaches the preset time, the process may return to the motor control step S200. Then, at the motor control step S200, the motor 162 may be driven, and the tissue may be compressed again. The preset time may be within a range of 10 to 20 seconds, and most preferably 15 seconds.

On the other hand, when the real-time distance Dₙ is within the set distance Dᵣ at the real-time pressure Pₙ and distance Dₙ measurement step S300, the process may proceed to the anastomosis unit control step S400. That is, when the real-time pressure Pₙ and the real-time distance Dₙ reach the set pressure Pᵣ and the set distance Dᵣ stored in advance, the anastomosis unit 130 is controlled at the anastomosis unit control step by the control unit 170 to cut unnecessary parts of the tissue and anastomose the tissue with a plurality of staples S400.

Here, in measuring in real-time the real-time pressure Pₙ applied to the tissue, the real-time pressure Pₙ may be calculated based on a previously stored current-rotation force-pressure proportion relation equation after the difference value between the real-time current value Iₙ, which is measured by the current sensor 171 in the control unit 170 according to the rotational force of the motor 162, and the driving current Iₘ is calculated.

Meanwhile, at the real-time pressure Pₙ and distance Dₙ measurement step S300, it is possible to amplify the real-time current value Iₙ, remove noise voltage, and convert a voltage input as an analog signal into a digital signal. In addition, the real-time pressure Pₙ may be calculated based on a previously stored current-rotation force-pressure proportion relation equation after the difference value between the preprocessed real-time current value Iₙ and the driving current Iₘ is calculated.

Here, the current-rotation force-pressure proportion relation equation is as shown in [Equation 1].

Pₙ=ηN_{T}_{_{d}}/A [Equation 1]

Here, Pₙ is a real-time pressure applied to the tissue, η is a power transmission efficiency coefficient, N is a gear ratio of the screw gear, and A is a contacting area between the bottom surface of the anvil 110 and the anastomosis 130.

T_{d} is a torque generated by the tissue and may be calculated using [Equation 2] shown below.

T_{d}=Kₜ/I_{d}=Kₜ(Iₙ−Iₘ) [Equation 2]

Here, Kₜ is the torque constant of the motor 162, I_{d} is the difference value between the real-time current value Iₙ and the driving current Iₘ, Iₘ is the driving current, and Iₙ is real-time current measured by the current sensor 171.

In addition, in measuring the real-time distance Dₙ between the top surface of the anastomosis unit 130 and the bottom surface of the anvil 110, the real-time pressure Pₙ and distance Dₙ measurement step S300 may include a pulse count acquisition step S310. At the pulse count acquisition step S310, the number of pulses generated in proportion to the rotation amount may be acquired by the distance calculating unit 173 in the control unit 170.

At the real-time pressure Pₙ and distance Dₙ measurement step S300, the real-time distance Dₙ may be determined using the number of pulses and the gear ratio of the screw gear 141. Most preferably, the real-time distance Dₙ may be calculated using [Equation 3] shown below.

$${D}_{n} = {D}_{0} + \frac{{K}_{e} {M}_{n}}{{N}_{g} {M}_{r}} \qquad [ Equation 3 ]$$

Here, D₀ is the minimum distance, Kₑ is the linear movement distance per rotation of the motor 162, Mₙ is the number of pulses acquired at the pulse count acquisition step S310, N_{g} is the gear ratio of the reducer of the motor 162, and Mᵣ is the number of pulses per rotation of the motor 162.

That is, at the real-time pressure Pₙ and distance Dₙ measurement step S300, the real-time distance Dₙ may be calculated by counting the number of pulses generated while the motor 162 rotates.

Therefore, according to the present invention, when a perforated tissue is anastomosed during laparoscopic surgery, as a real-time pressure Pₙ applied to the tissue and a real-time distance Dₙ between the top surface of the anastomosis unit 130 and the bottom surface of the anvil 110 can be grasped, it is possible to reduce damage to a compression surface between tissues that a surgeon does not recognize with naked eyes, and reduce the prevalence rate of complications by preventing over-compression or under-compression of the tissues.

As described above, although the embodiments have been described with reference to the limited embodiments and drawings, various modifications and variations are possible from the above description by those skilled in the art. For example, an appropriate result may be achieved although the described techniques are performed in an order different from those of the described method, and/or the described components of the system, structure, device, circuit, and the like are coupled or combined in a form different from those of the described method, or replaced or substituted by other components or equivalents.

Therefore, other implementations, other embodiments, and equivalents to the claims are also within the scope of the claims described below.

According to the present invention as described above, as an automatic compression circular anastomosis device comprising an anvil, a separation coupling unit, an anastomosis unit, a shaft, a handle unit, a body, and a control unit is provided, and a real-time pressure Pₙ applied to a tissue and a real-time distance Dₙ between the top surface of the anastomosis unit and the bottom surface of the anvil are grasped, there is an effect of reducing damage to a compression surface between tissues that a surgeon does not recognize with naked eyes and significantly reducing the prevalence rate of complications by preventing over-compression or under-compression of the tissues when a perforated tissue is anastomosed during laparoscopic surgery.

### DESCRIPTION OF SYMBOLS

100: Automatic compression circular anastomosis device
110: Anvil
120: Separation coupling unit
121: Protruding stopper
130: Anastomosis unit
131: Receiving groove stopper
140: Shaft
141: Screw gear
150: Handle unit
151: Operation switch
152: Upper handle
160: Body
161: Bearing
162: Motor
163: Couple ring
164: Power transmission adapter
165: Encoder
166: Emergency switch
170: Control unit
171: Current sensor
172: Pressure calculation unit
173: Distance calculation unit

What is claimed is:

1. An automatic compression circular anastomosis device comprising:
an anvil formed in a conical shape to be easily inserted into a perforated tissue;
a separation coupling unit having a central axis formed in a direction from a bottom surface of the anvil toward the outside;
an anastomosis unit for pressing, when the tissue is positioned around the separation coupling unit, both sides of the tissue together with the bottom surface of the anvil, cutting unnecessary parts of the tissue, and anastomosing the tissue with a plurality of staples;
a shaft bent to be easily inserted into the perforated tissue and provided with a screw gear for converting a rotational force of the motor into an axial linear movement to allow the separation coupling unit coupled to the anastomosis unit to move linearly to transfer power;
a handle unit formed to accommodate one side of the shaft and gripped by a user to generate a pressing force by an external force of the user;
a body including a bearing having a shaft hole, the motor having a power transmission shaft passing through the shaft hole on one side and generating a rotational force, a couple ring locked and fixed to the power transmission shaft between one side of the bearing and the motor not to generate eccentricity, a power transmission adapter located on the other side of the bearing to transmit the rotational force of the motor to the screw gear, an encoder having a rotating shaft coupled on the other side of the motor and measuring an amount of rotation of the rotating shaft, and an emergency switch for immediately cutting off the power applied to a control unit; and
the control unit including a current sensor for measuring a current value according to the rotational force of the motor, a pressure calculation unit for calculating a real-time pressure Pₙ applied to the tissue using the current value, and a distance calculation unit for calculating a distance between a top surface of the anastomosis unit and the bottom surface of the anvil using the rotation amount measured by the encoder, and controlling the motor according to a set pressure Pᵣ and a set distance Dᵣ stored in advance.

2. A control method of an automatic compression circular anastomosis device, the method comprising:
a correction step of correcting a zero point when an operation switch provided in a handle unit is turned on, by a control unit;
a motor control step of controlling a motor to compress a tissue in a direction reaching a minimum distance D₀ at which a distance between a top surface of an anastomosis unit and a bottom surface of an anvil is minimized, by the control unit;
a real-time pressure Pₙ and distance Dₙ measurement step of measuring a real-time pressure Pₙ applied to the tissue and a real-time distance Dₙ between the top surface of the anastomosis unit and the bottom surface of the anvil in real-time, by the control unit; and
an anastomosis unit control step of controlling, when the real-time pressure Pₙ and the real-time distance Dₙ reach a set pressure Pᵣ and a set distance Dᵣ stored in advance, the anastomosis unit to cut unnecessary parts of the tissue and anastomose the tissue with a plurality of staples, by the control unit.

3. The method according to claim 2, wherein the correction step includes:
a zero-point storage step of storing, when the motor rotates in a direction reaching the minimum distance D₀ and a current value I measured by a current sensor exceeds a previously stored set current Iᵣ, the corresponding current value I and a corresponding point as a zero-point current I₀ and the zero point; and
a driving current Iₘ measurement step of measuring a driving current Iₘ at each point while the motor rotates in a direction reaching the minimum distance D₀ again after the motor rotates as much as a predetermined distance in a direction reaching the maximum distance D₁ at which the distance between the top surface of the anastomosis unit and the bottom surface of the anvil is maximized at the zero point, by the control unit.

4. The method according to claim 3, wherein at the real-time pressure Pₙ and distance Dₙ measurement step, after a difference value between the real-time current value Iₙ measured by the current sensor according to a rotational force of the motor and the driving current Iₘ is calculated, the real-time pressure Pₙ is calculated based on a previously stored current-rotation force-pressure proportion relation equation.

5. The method according to claim 2, wherein the real-time pressure Pₙ and distance Dₙ measurement step includes a pulse count acquisition step of acquiring the number of pulses generated in proportion to the rotation amount, wherein the real-time distance Dₙ is determined using the number of pulses and a gear ratio of a screw gear.
